(19) United States
(12) Patent Application Publication
(10) Pub. No.: US 2026/0299734 A1
(43) Pub. Date: Oct. 1, 2026

(54) PROPRIOCEPTION MEASUREMENT SYSTEM

(71) Applicant: KOREA INSTITUTE OF SCIENCE AND TECHNOLOGY, Seoul (KR)

(72) Inventors: Byounghyun YOO, Seoul (KR); Gun Yeol NA, Seoul (KR)

(73) Assignee: KOREA INSTITUTE OF SCIENCE AND TECHNOLOGY, Seoul (KR)

(21) Appl. No.: 19/447,574

(22) Filed: Jan. 13, 2026

(30) Foreign Application Priority Data
Mar. 31, 2025 (KR) 10-2025-0041288

(51) Int. Cl. G06F 3/041; A61B 5/00; G09G 3/38

(52) U.S. Cl. CPC G06F 3/04186; A61B 5/4023; A61B 5/7435; G09G 3/38; G09G 2320/0233; G09G 2320/0666; G09G 2354/00

(57) ABSTRACT

A proprioception measurement system includes a transparent display panel having an upper surface and configured to display a target point, which is visually recognizable by a subject, on the upper surface, and a transparent touch panel disposed on a lower surface of the transparent display panel and configured to detect a reaching point, which is on a lower surface of the transparent display panel and touched by the subject to reach a target point displayed on the upper surface of the transparent display panel, and accordingly the subject's proprioception may be accurately measured without proprioception extension phenomenon.

### CROSS-REFERENCE TO RELATED APPLICATION

This application is based on and claims priority under 35 U.S.C. §119 to Korean Patent Application No. 10-2025-0041288, filed on Mar. 31, 2025, in the Korean Intellectual Property Office, the disclosure of which is incorporated by reference herein in its entirety.

### BACKGROUND

### Field

The disclosure relates to a system for measuring the degree of functional degradation of proprioception.

### Description of the Related Art

Visual recognition is the sense that detects light in the external environment through eyes and interprets the detected light to recognize an object's shape, color, distance, and movement. Proprioception is the sense that detects a body's position, movement, force, and balance through sensory receptors located in muscles, joints, and tendons. By combining visual recognition and proprioception, humans may effectively control bodies thereof and move stably. For example, a person may combine visual information on visually recognized keys of a keyboard with information on a position and movement of muscles, joints, and tendons of hands perceived through proprioception to appropriately control the hand’s movement to press the keys as intended.

When a person who uses a keyboard by combining visual recognition and proprioception loses sight of the hand, the person has to rely solely on proprioception to estimate a position and movement of the hand. In this case, when proprioceptive function is impaired, a person may press unintended keys. Such a functional degradation of proprioception may lead to a decrease in postural control, protective reflexes, joint movement, and balance. An indicator representing the functional degradation of proprioception, such as visual-proprioceptive matching error, may be an indication of balance disorder, impaired postural control, and neurological disorder. Therefore, accurate measurement of proprioception is required.

Measuring a visual-proprioceptive matching error is generally performed by having a subject visually perceive a position of a target point and manually move a cursor to the target point. The visual-proprioceptive matching error may be measured from a difference between a position of a target point and a position of a reaching point that is a cursor's position. The conventional method for measuring proprioception includes a method of analyzing a video in which positions of a target point and a reaching point are recorded, a method of using a separate input device, such as a stylus, as a cursor, and so on. The former method hinders accurate measurement due to a limited resolution of the video and the latter method may introduce errors in proprioceptive measurement results due to a long distance between the target point and the reaching point, which causes proprioceptive sensation to expand.

### SUMMARY

The disclosure provides a proprioception measurement system that accurately measure a subject's proprioception by preventing a phenomenon of proprioception expansion in a subject. The technical issues described above are not limited thereto, and other technical issues may be derived from following descriptions.

According to an aspect of the disclosure, a proprioception measurement system includes a transparent display panel configured to display a target point, which is visually recognizable by a subject, on an upper surface of the transparent display panel, a transparent touch panel disposed under a lower surface of the transparent display panel and configured to detect a reaching point, which is touched toward a lower surface of the transparent display panel by the subject to reach a target point displayed on the upper surface of the transparent display panel, and a computer configured to measure proprioception of the subject based on a display position of the target point displayed on the upper surface of the transparent display panel and a detection position of the reaching point detected by the transparent touch panel.

The transparent touch panel may have two surfaces, one surface of which having a touch detection function may be disposed facing downward under the lower surface of the transparent display panel, such that a hand of the subject is located in a lower space of the transparent touch panel to input the reaching point to the transparent touch panel in a state where both eyes of the subject look at the upper surface of the transparent display panel.

A point, which is touched by the subject so as to reach the target point simultaneously using both visual recognition and the proprioception of the subject in a state where a surrounding region of the target point is displayed in a transparent color such that a movement of a hand of the subject located below the transparent touch panel is visually observed by the subject, may be input to the transparent touch panel as the reaching point.

A point, which is touched by the subject so as to reach the target point using proprioception of the subject in a state where the surrounding region of the target point is displayed in an opaque color such that the movement of the hand of the subject located below the transparent touch panel is not visually observed by the subject, may be input to the transparent touch panel as the reaching point.

The transparent display panel may have a multilayer structure including a pixel layer composed of multiple pixels, a transparent electrode layer configured to supply power to the multiple pixels, and a transparent substrate layer, and the surrounding region of the target point may be displayed in the transparent color or the opaque color according to color transparency of each of the multiple pixels corresponding to the surrounding region of the target point among the multiple pixels of the pixel layer.

The proprioception measurement system may further include an electrochromic glass plate disposed on the upper surface or the lower surface of the transparent display panel and configured to change in transparency according to a voltage applied to the electrochromic glass plate, wherein the surrounding region of the target point may be displayed in the transparent color or the opaque color according to the transparency of the electrochromic glass plate.

The proprioception measurement system may further include a projector configured to project light toward a position which is preset by a user for an upper surface of the electrochromic glass plate, wherein the target point may be formed and displayed on the upper surface of the electrochromic glass plate by the light projected from the projector in a state where the electrochromic glass plate is opaque.

The proprioception measurement system may further include an opaque film disposed on the upper surface of the transparent display panel or disposed outside a two-dimensional display region of the transparent display panel, wherein the surrounding region of the target point may be displayed in the transparent color or the opaque color depending on disposition positions of the opaque film.

The proprioception measurement system may further include a projector configured to project light toward a position that is preset by a user for an upper surface of the opaque film, wherein the target point may be formed and displayed on the upper surface of the opaque film by the light projected from the projector in a state where the opaque film is disposed on the upper surface of the transparent display panel.

The computer may be further configured to output an error in a detection position of the target point detected by the transparent touch panel relative to a display position of the target point displayed on the upper surface of the transparent display panel as a measurement result of the proprioception of the subject.

The proprioception measurement system may further include a housing formed in a shape of a quadrangular box having an open upper surface and an open front surface and configured to provide a space for a lower surface of the transparent touch panel to be touched by a hand of the subject, wherein the transparent touch panel may be mounted on the open upper surface of the housing such that the transparent touch panel is touched by the hand of the subject located in an internal space of the housing through the open front surface of the housing.

The proprioception measurement system may further include a height adjustment member installed below a lower surface of the housing to adjust a height of the lower surface of the housing depending on the height of the subject.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a configuration view of a proprioception measurement system according to an embodiment of the disclosure;

FIG. 2 is an exploded view of the proprioception measurement system illustrated in FIG. 1;

FIG. 3 is a detailed exploded view of the proprioception measurement system illustrated in FIG. 1;

FIGS. 4 and 5 illustrate examples of inputting a reaching point to the proprioception measurement systems illustrated in FIGS. 1 to 3;

FIG. 6 is a configuration view of a proprioception measurement system according to another embodiment of the disclosure;

FIG. 7 is an exploded view of the proprioception measurement system illustrated in FIG. 6.;

FIG. 8 is a configuration view of a proprioception measurement system according to another embodiment of the disclosure;

FIG. 9 is an exploded view of the proprioception measurement system illustrated in FIG. 8;

FIGS. 10 and 11 illustrate examples of inputting a reaching point to the proprioception measurement systems illustrated in FIGS. 8 and 9;

FIG. 12 is a configuration view of a proprioception measurement system according to another embodiment of the disclosure;

FIG. 13 is an exploded view of the proprioception measurement system illustrated in FIG. 12; and

FIGS. 14 and 15 are examples of inputting a reaching point to the proprioception measurement system illustrated in FIGS. 12 and 13.

### DETAILED DESCRIPTION

Hereinafter, embodiments of the disclosure will be described in detail with reference to the drawings. Embodiments of the disclosure below relates to a proprioception measurement system that enables accurate measurement of a subject's proprioception by preventing a phenomenon of proprioception expansion in the subject, which are referred to simply as a "proprioception measurement system". Hereinafter, a target person whose proprioception is measured by a proprioception measurement system according to embodiments is referred to simply as a "subject", and a user of the proprioception measurement system is referred to simply as a "user." A user may be a subject or may be someone other than the subject, such as a doctor.

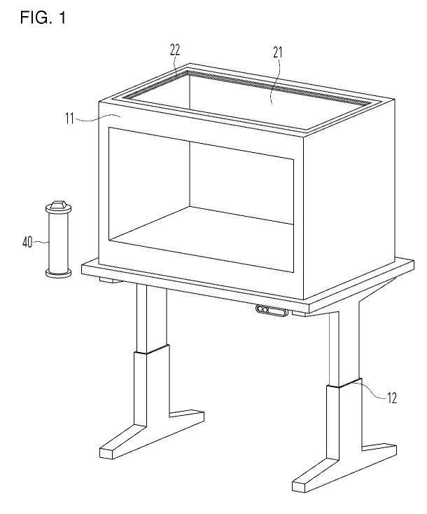

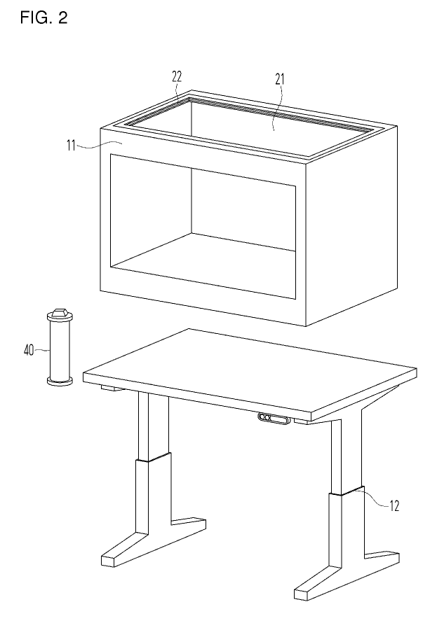

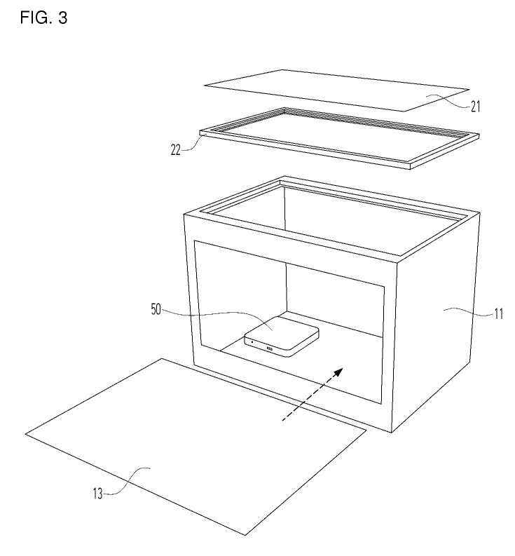

FIG. 1 is a configuration view of a proprioception measurement system according to an embodiment of the disclosure, FIG. 2 is an exploded view of the proprioception measurement system illustrated in FIG. 1, and FIG. 3 is a partial detailed exploded view of the proprioception measurement system illustrated in FIG. 1. Referring to FIGS. 1 to 3, the proprioception measurement system illustrated in FIGS. 1 to 3 include a housing 11, a height adjuster 12, a transparent display panel 21, a transparent touch panel 22, a reaching point determination input unit 40, and a computer 50. Those skilled in the art will understand that other configurations may be added to the configuration described above.

The housing 11 is formed in the form of a quadrangular box having an open upper surface and an open front surface, serves to support the transparent display panel 21 and the transparent touch panel 22, and provides a space for a lower surface of the transparent touch panel 22 to be touched by a subject. As illustrated in FIGS. 1 to 3, a left surface, a right surface, a lower surface, and a rear surface of the housing 11 are closed, and an upper surface and a front surface are open. The transparent touch panel 22 is mounted on the open upper surface of the housing 11 such that the transparent touch panel 22 may be touched by the hand of a subject located in an internal space of the housing 11 through the open front surface of the housing 11. The transparent touch panel 22 is mounted on the open upper surface of the housing 11 in the manner in which the transparent touch panel 22 is mounted on an upper edge of the housing 11 forming the open upper surface of the housing 11 such that one surface of the transparent touch panel 22 having a touch detection function faces a lower side.

The height adjuster 12 is installed on a lower surface of the housing 11 and adjusts a height of a lower surface of the housing 11 according to the height of a subject. The height adjuster 12 may be implemented as a motion desk. As illustrated in FIGS. 1 and 2, the height adjuster 12 includes a top plate on which the housing 11 may be placed, two legs supporting the top plate, an actuator for adjusting lengths of the two legs, and a user interface for receiving user input. Each of the two legs is formed to fit each of two pipes. The actuator includes a motor, a gear, and so on, is installed inside each of the two pipes, and functions to lift and drop an upper pipe of each leg while being fitted to a lower pipe.

The transparent display panel 21 displays a target point, which may be visually recognized by a subject at a target point preset by a user on an upper surface of the transparent display panel 21 under the control of the computer 50, in an opaque color, and displays a surrounding region of the target point in a transparent color or an opaque color. For example, the target point may be displayed as an opaque blue dot. A transparent color of the transparent display panel 21 refers to a color within a transparency range in which a subject's hand movement located below a lower surface of the transparent display panel 21 may be visually recognized by the subject when the subject's eyes are located above the transparent display panel 21. An opaque color of the transparent display panel 21 refers to a color within a transparency range in which a subject's hand movement located below the lower surface of the transparent display panel 21 may not be visually recognized by the subject when the subject's eyes are located above the transparent display panel 21. The transparent or opaque color of the transparent display panel 21 may be set in various ways.

The transparent display panel 21 illustrated in FIGS. 1 to 3 may be implemented by a liquid crystal display (LCD) transparent display or an organic light emitting diode (OLED) transparent display. The transparent display panel 21 implemented in an LCD or OLED transparent display has a multilayer structure including a pixel layer composed of multiple pixels, a transparent electrode layer supplying power to the multiple pixels, a transparent substrate layer, and so on. The surrounding region of the target point is displayed in a transparent or opaque color according to the color transparency of each of the multiple pixels corresponding to the surrounding region of the target point among the multiple pixels in the pixel layer of the transparent display panel 21.

The transparent touch panel 22 is disposed below a lower surface of the transparent display panel 21 and detects a reaching point, which is a point where the transparent touch panel 22 is touched by a subject toward the lower surface of the transparent display panel 21 so as to reach the target point displayed on an upper surface of the transparent display panel 21. The transparent touch panel 22 may be an infrared touch panel that detects the touch of a subject's finger by using infrared light. The transparent touch panel 22 is disposed under the lower surface of the transparent display panel 21 to enable one surface having a touch detection function among two surfaces of the transparent touch panel 22 to face downward such that a subject's hand is located in a lower space of the transparent touch panel 22 to input the target point to the transparent touch panel 22 in a state where both eyes of the subject look at an upper surface of the transparent display panel 21. In the embodiment illustrated in FIGS. 1 to 3, the transparent touch panel 22 is disposed under a lower surface of the transparent display panel 21. It is preferable that the transparent display panel 21 and the transparent touch panel 22 be installed parallel to the ground.

The reaching point determination input unit 40 generates a reaching point determination signal for determining one of multiple points of the transparent touch panel 22 touched by a subject as a reaching point at a point in time according to the subject's operation, and transmits the generated reaching point determination signal to the computer 50. The reaching point determination signal may be transmitted from the reaching point determination input unit 40 to the computer 50 using various short-range communication methods such as Bluetooth and Wi-Fi. As illustrated in FIGS. 1 and 2, the reaching point determination input unit 40 may have a structure in which a button is attached to the top of a rod-shaped body that may be easily held in a hand of a subject. The reaching point determination input unit 40 generates the reaching point determination signal when the button is pressed by a subject and transmits the generated reaching point determination signal to the computer 50. As described below, the reaching point determination input unit 40 may not be used depending on methods of determining a detection position of a reaching point detected by the transparent touch panel 22.

The computer 50 controls the transparent display panel 21 such that, according to a user's operation, the transparent display panel 21 displays a target point and displays a surrounding region of the target point in a transparent or opaque color. The computer 50 measures a subject's proprioception based on a display position of the target point displayed on an upper surface of the transparent display panel 21 and a detection position of the reaching point detected by the transparent touch panel 22. As illustrated in FIG. 3, the computer 50 may be disposed in an internal space of the housing 11. A protective plate 13 that may cover the computer 50 may be installed in an internal space of the housing 11 to protect the computer 50 disposed therein. When a subject touches a lower surface of the transparent touch panel 22 to reach the target point displayed on an upper surface of the transparent display panel 21, the lower surface of the transparent touch panel 22 may be touched multiple times. The detection position of the reaching point detected by the transparent touch panel 22 may be determined in various ways as follows.

The computer 50 may determine a detection position of the point touched by a subject as a detection position of the reaching point detected by the transparent touch panel 22 at a point in time when receiving a reaching point determination signal from the reaching point determination input unit 40 among multiple points of the transparent touch panel 22 touched by the subject. The computer 50 may determine an arithmetic average position of the detection position of multiple points of the transparent touch panel 22 touched by a subject during a preset period of time as the detection position of the reaching point detected by the transparent touch panel 22, or may determine a detection position of the last touched point among the multiple points of the transparent touch panel 22 touched by the subject during the preset period of time as the detection position of the reaching point detected by the transparent touch panel 22.

The computer 50 may output an error of the detection position of the reaching point detected by the transparent touch panel 22 with respect to a display position of the target point displayed on an upper surface of the transparent display panel 21 as a measurement result of a subject's proprioception. A two-dimensional display space corresponding to an upper surface of the transparent display panel 21 and a two-dimensional detection space corresponding to a lower surface of the transparent touch panel 22 may be expressed as the same two-dimensional coordinate system. As an error in the detection position of the target point detected by the transparent touch panel 22 with respect to the display position of the target point displayed on an upper surface of the transparent display panel 21, the computer 50 may calculate a distance between a coordinate value (x,y) of the display position of the target point displayed on an upper surface of the transparent display panel 21 and a coordinate value (x,y) of the detection position of the reaching point detected by the transparent touch panel 22.

The computer 50 may be a mini personal computer (PC) illustrated in FIG. 3. An algorithm for measuring a subject's proprioception by displaying a target point on a transparent display panel 21 according to a user's operation and displaying a surrounding region of the target point in a transparent or opaque color is written as a computer program and stored in the computer 50. A target point may be displayed on an upper surface of the transparent display panel 21 and a surrounding region of the target point may be displayed in a transparent or opaque color by controlling a liquid crystal voltage of the transparent display panel 21 or a current of an OLED according to an operation of a computer program. The computer 50 may output a measurement result of a subject's proprioception onto the transparent display panel 21 as text, images, or so on.

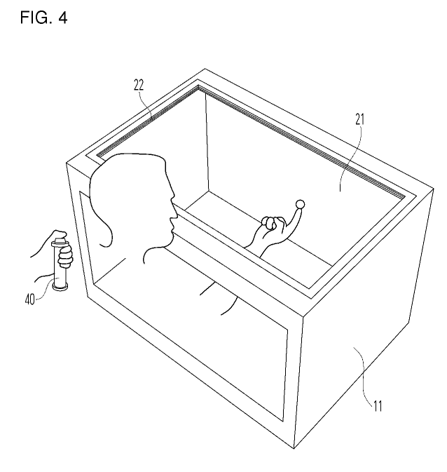

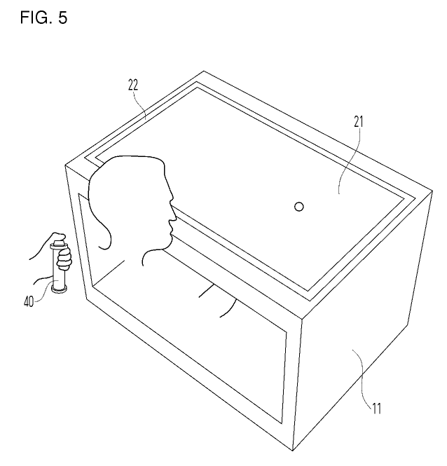

FIGS. 4 and 5 are examples of inputting a reaching point to the proprioception measurement system illustrated in FIGS. 1 to 3. FIG. 4 illustrates a scene where a reaching point is input to the transparent touch panel 22 by a subject while a surrounding region of a target point displayed on the transparent display panel 21 is transparent. In a state where the surrounding region of the target point is displayed in a transparent color on the transparent display panel 21 such that a subject may visually observe the movement of the subject's hand located below the transparent touch panel 22, a point touched by the subject is input as the reaching point to the transparent touch panel 22 so as to reach the target point using both a subject's vision and proprioception. In this case, a subject puts his/her hand into the housing 11 and touches the transparent touch panel 22 such that a fingertip reaches a target point while observing the movement of the fingertip using both visual information on his/her hand and proprioception. In this case, when a subject presses a button of the reaching point determination input unit 40, a reaching point determination signal is transmitted to the computer 50.

FIG. 5 illustrates a scene where a reaching point is input to the transparent touch panel 22 by a subject in a state where a surrounding region of the target point displayed on the transparent display panel 21 is opaque. In a state where the surrounding region of the target point is displayed in an opaque color on the transparent display panel 21 such that the movement of a hand of a subject located below the transparent touch panel 22 may not be visually observed by the subject, the subject may not observe the movement of a finger, and accordingly, a point touched by the subject is input as the reaching point to the transparent touch panel 22 such that the subject reaches the target point using the subject's proprioception. In this case, the subject puts his/her hand into the housing 11 and touches the transparent touch panel 22 such that the subject’s fingertip reaches the target point using only proprioception of the subject’s hand in a state where visual information on the subject’s hand is blocked. In this case, when a button of the reaching point determination input unit 40 is pressed by a subject, a reaching point determination signal is transmitted to the computer 50. The mismatch between vision and proprioception may be measured from a transparent color of the transparent display panel 21 illustrated in FIG. 4, while the degree of functional degradation of proprioception alone may be measured from an opaque color of the transparent display panel 21 illustrated in FIG. 5.

In the conventional proprioception measurement method, a device for displaying a target point is separated from a device for receiving a reaching point, and accordingly, proprioception corresponding to a distance between the target point and the reaching point may be expanded. As a result, a proprioception measurement result may cause an error due to the expansion of proprioception. According to the embodiment illustrated in FIGS. 1 to 5, the transparent touch panel 22 is disposed under a lower surface of the transparent display panel 21, a reaching point, which is a point touched by a subject toward a lower surface of the transparent display panel 21 to reach a target point displayed on an upper surface of the transparent display panel 21, is detected, and accordingly, an proprioception expansion phenomenon of the subject is hardly generated.

In this way, in the embodiment illustrated in FIGS. 1 to 5, the proprioception of a subject may be accurately measured because the proprioception expansion phenomenon of the subject is hardly generated. As illustrated in FIGS. 1 to 5, the transparent touch panel 22 is disposed to overlap a lower surface of the transparent display panel 21, and thicknesses of the transparent display panel 21 and the transparent touch panel 22 are thinner, and accordingly, a reaching point is close to a target point. In addition, as resolutions of the transparent display panel 21 and the transparent touch panel 22 are improved, an error detection accuracy of the reaching point detected by the transparent touch panel 22 with respect to a display position of the target point displayed on an upper surface of the transparent display panel 21 is increased, and accordingly, a measurement accuracy of a subject's proprioception may be easily designed to suit a utilization purpose of the measurement result.

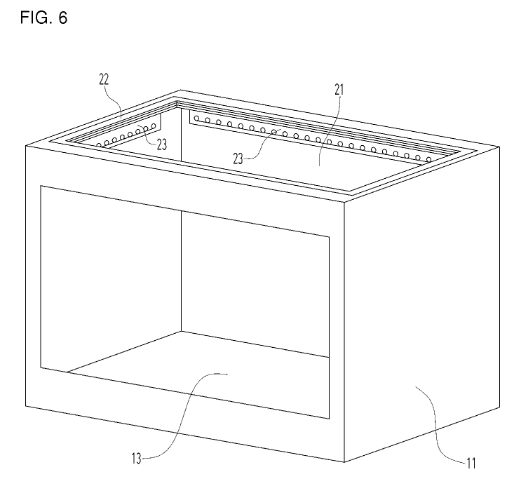

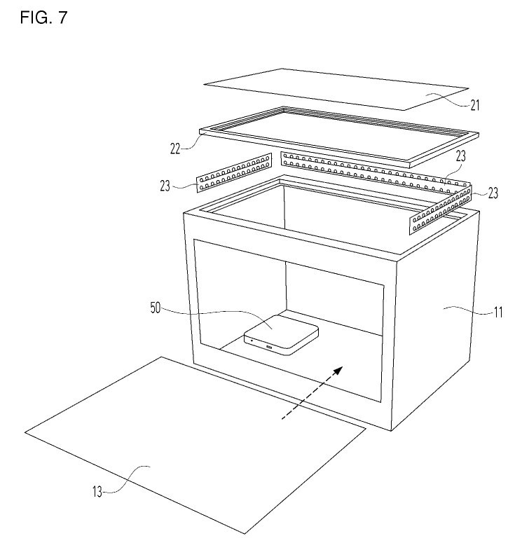

FIG. 6 is a configuration view of a proprioception measurement system according to another embodiment of the disclosure, and FIG. 7 is an exploded view of the proprioception measurement system illustrated in FIG. 6. Referring to FIGS. 6 and 7, a proprioception measurement system illustrated in FIGS. 6 and 7 includes a housing 11, a height adjuster 12, a transparent display panel 21, a transparent touch panel 22, a backlight unit 23, a reaching point determination input unit 40, and a computer 50. The proprioception measurement system illustrated in FIGS. 6 and 7 is identical to the proprioception measurement system illustrated in FIGS. 1 to 3, except that the backlight unit 23 is added to the proprioception measurement system illustrated in FIGS. 1 to 3. Accordingly, descriptions of the embodiment illustrated in FIGS. 6 and 7 are replaced with the descriptions of the embodiment illustrated in FIGS. 1 to 3, and a difference between the embodiment illustrated in FIGS. 6 and 7 and the embodiment illustrated in FIGS. 1 to 3 is described below.

When the transparent display panel 21 is an LCD transparent display, the backlight unit 23 may be additionally installed. The backlight unit 23 is installed below the transparent touch panel 22 disposed under a lower surface of the transparent display panel 21 and emits backlight to the transparent display panel 21. As illustrated in FIGS. 6 and 7, the backlight unit 23 may be attached to an upper inner surface of the housing 11 along a perimeter of the transparent display panel 21 in the form of a band to which multiple LEDs are attached. A diffusion sheet and so on may be additionally installed to diffuse the light from the multiple LEDs to provide uniform illumination to the entire transparent display panel 21.

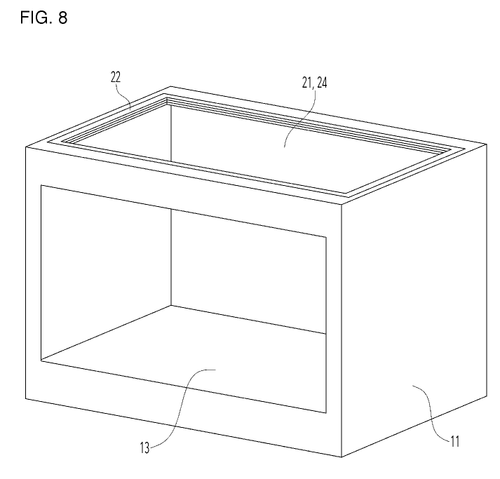

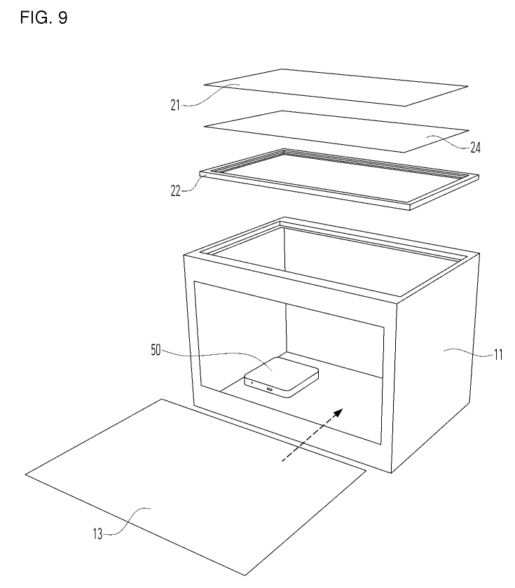

FIG. 8 is a configuration view of a proprioception measurement system according to another embodiment of the disclosure, and FIG. 9 is an exploded view of the proprioception measurement system illustrated in FIG. 8. Referring to FIGS. 8 and 9, the proprioception measurement system illustrated in FIGS. 8 and 9 includes a housing 11, a height adjuster 12, a transparent display panel 21, a transparent touch panel 22, an electrochromic glass plate 24, a reaching point determination input unit 40, and a computer 50. The proprioception measurement system illustrated in FIGS. 8 and 9 is identical to the proprioception measurement system illustrated in FIGS. 1 to 3, except that the electrochromic glass plate 24 is added to the proprioception measurement system illustrated in FIGS. 1 to 3. Accordingly, descriptions of the embodiment illustrated in FIGS. 8 and 9 are replaced with the descriptions of the embodiments illustrated in FIGS. 1 to 3, and a difference between the embodiment illustrated in FIGS. 8 and 9 and the embodiment illustrated in FIGS. 1 to 3 is described below.

The electrochromic glass plate 24 is disposed on an upper surface or lower surface of the transparent display panel 21, and transparency of the electrochromic glass plate 24 changes according to a voltage applied thereto under the control of the computer 50. A surrounding region of a target point is displayed in a transparent or opaque color according to transparency of the electrochromic glass plate 24 disposed on the upper surface or lower surface of the transparent display panel 21. The electrochromic glass plate 24 is normally transparent because a metal oxide is deposited thereon, but when voltage is applied thereto, the electrochromic glass plate 24 is opaque. FIGS. 8 and 9 illustrate examples in which the electrochromic glass plate 24 is disposed on a lower surface of the transparent display panel 21. In this case, the transparent touch panel 22 is disposed on a lower surface of the electrochromic glass plate 24. When the electrochromic glass plate 24 is disposed on an upper surface of the transparent display panel 21, the transparent touch panel 22 is disposed under a lower surface of the transparent display panel 21.

The computer 50 may output a pixel layer control signal representing an image in which a target point is displayed on a transparent or opaque background to the transparent display panel 21 to control the transparent display panel 21 such that the transparent display panel 21 displays the target point and displays a surrounding region of the target point in a transparent or opaque color. When the pixel layer control signal representing the image in which the target point is displayed on the opaque background is input to the transparent display panel 21, pixels corresponding to the surrounding region of the target point display an opaque color. In the embodiments illustrated in FIGS. 1 to 3, when opacity of the surrounding region of the target point displayed on the transparent display panel 21 is insufficient, a subject's hand movement may be observed blurry by the subject.

In order to accurately measure the degree of functional degradation of proprioception in an opaque color of the transparent display panel 21, a subject's visual recognition on the subject's hand movement has to be completely blocked. The electrochromic glass plate 24 is disposed on an upper surface or lower surface of the transparent display panel 21 to complement for an opaque color of the transparent display panel 21, and accordingly, a subject's visual recognition on the subject's hand movement may be completely blocked. When the transparent display panel 21 and the electrochromic glass plate 24 overlap each other, opacity of the electrochromic glass plate 24 accumulates on opacity of the transparent display panel 21, and accordingly. the entire opacity increases.

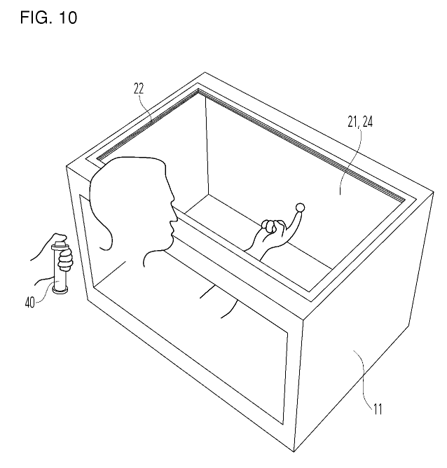

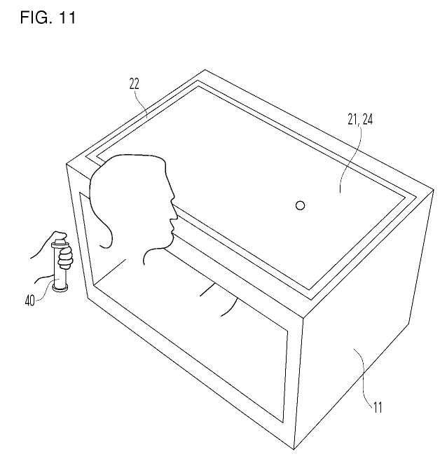

FIGS. 10 and 11 are examples of inputting a reaching point to the proprioception measurement system illustrated in FIGS. 8 and 9. FIG. 10 illustrates a state where a surrounding region of a target point displayed on the transparent display panel 21 is transparent and a reaching point is input to the transparent touch panel 22 by a subject while the electrochromic glass plate 24 is transparent. A point touched by a subject is input to the transparent touch panel 22 as a reaching point to reach a target point by simultaneously using the subject's visual recognition and proprioception in a state where a surrounding region of the target point is displayed in a transparent color on the transparent display panel 21 and the electrochromic glass plate 24 is in a transparent state such that a movement of the subject's hand located below the transparent touch panel 22 may be visually observed by the subject. In this case, a subject puts his/her hand into the housing 11 and touches the transparent touch panel 22 such that a fingertip reaches a target point while observing a movement of the fingertip utilizing both visual information and proprioception on his/her hand.

FIG. 11 illustrates a scene where a reaching point is input to the transparent touch panel 22 by a subject in a state where a surrounding region of a target point displayed on the transparent display panel 21 is opaque and the electrochromic glass plate 24 is opaque. Because a subject may not observe a finger movement in a state where a surrounding region of a target point is displayed in an opaque color on the transparent display panel 21 and the electrochromic glass plate 24 is opaque such that a movement of the subject's hand located below the transparent touch panel 22 may not be visually observed by the subject, a point touched by the subject is input to the transparent touch panel 22 as a reaching point to reach the target point using the subject's proprioception. In this case, the subject puts his/her hand into the housing 11 and touches the transparent touch panel 22 such that a fingertip reaches the target point utilizing only the proprioception of his/her hand while visual information on his/her hand is blocked.

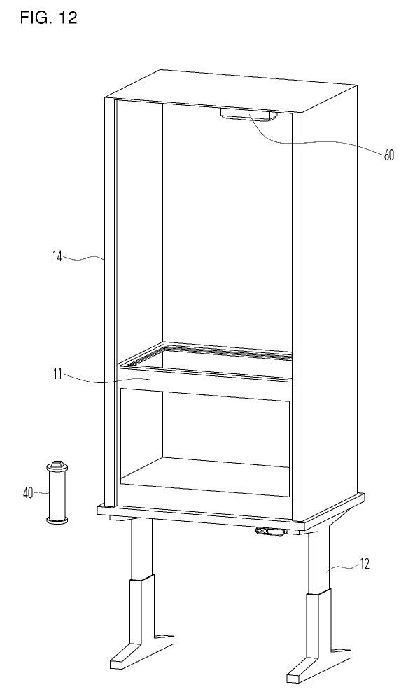

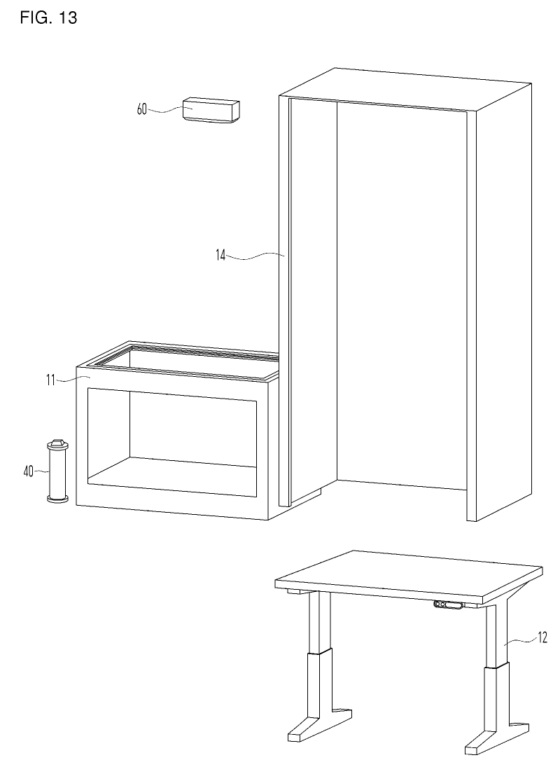

FIG. 12 is a configuration view of a proprioception measurement system according to another embodiment of the disclosure, and FIG. 13 is an exploded view of the proprioception measurement system illustrated in FIG. 12. Referring to FIGS. 12 and 13, the proprioception measurement system illustrated in FIGS. 12 and 13 includes a housing 11, a height adjuster 12, an upper scene, a transparent display panel 21, a transparent touch panel 22, an electrochromic glass plate 24, a reaching point determination input unit 40, a computer 50, and a plate 24. The proprioception measurement system illustrated in FIGS. 12 and 13 is identical to the proprioception measurement system illustrated in FIGS. 1 to 3, except that the upper 14, the electrochromic glass plate 24, and the projector 60 are added to the proprioception measurement system illustrated in FIGS. 1 to 3. Accordingly, descriptions of the embodiment illustrated in FIGS. 12 and 13 are replaced with descriptions of the embodiment illustrated in FIGS. 1 to 3, and a differences between the embodiments illustrated in FIGS. 12 and 13 and the embodiment illustrated in FIGS. 1 to 3 is described below.

Similarly to the embodiment illustrated in FIGS. 8 and 9, the electrochromic glass plate 24 is disposed on an upper surface or lower surface of the transparent display panel 21, and transparency of the electrochromic glass plate 24 changes according to a voltage applied thereto under the control of the computer 50. A surrounding region of a target point is displayed in a transparent or opaque color according to transparency of the electrochromic glass plate 24 disposed on the upper surface or lower surface of the transparent display panel 21. Further description of the electrochromic glass plate 24 is replaced with the description of the embodiment illustrated in FIGS. 8 and 9. In a case where the electrochromic glass plate 24 is disposed on an upper surface of the transparent display panel 21, when opacity of the electrochromic glass plate 24 is higher, a target point displayed on the upper surface of the transparent display panel 21 may be covered by the electrochromic glass plate 24, and accordingly, it may be difficult for a subject to identify the target point.

In a state where the electrochromic glass plate 24 is disposed on an upper surface of a transparent display panel 21 and the electrochromic glass plate 24 is opaque, the projector 60 projects light toward a position, which is preset by a user for an upper surface of the electrochromic glass plate 24, under the control of the computer 50, and accordingly, a target point is formed on the upper surface of the electrochromic glass plate 24. In a state where the electrochromic glass plate 24 is disposed on the upper surface of the transparent display panel 21 and the electrochromic glass plate 24 is opaque, a target point is formed on the upper surface of the electrochromic glass plate 24 by the light projected from the plate 24, and accordingly, the target point that may be visually recognized by a subject may be displayed at a position set by a user. In this case, it is preferable that the transparent display panel 21 displays a surrounding region of a target point in an opaque color to increase the entire opacity. The transparent display panel 21 may or may not display a target point, and may be turned off when the opacity of the electrochromic glass plate 24 is higher.

Even when the electrochromic glass plate 24 is disposed on a lower surface of the transparent display panel 21, the projector 60 projects light toward a position, which is preset by a user for an upper surface of the transparent display panel 21, under the control of the computer 50 such that a target point may be clearly identified by a subject, and accordingly, the target point may also be formed on the upper surface of the transparent display panel 21. By forming a target point on an upper surface of the transparent display panel 21 according to the light projected from the plate 24, the target point, which may be visually recognized by a subject, may be displayed at a position preset by a user. In this case, it is preferable that the transparent display panel 21 displays a target point and displays a surrounding region of the target point in an opaque color. The projector 60 may be a beam projector or a laser projector.

The computer 50 controls the transparent display panel 21 such that the transparent display panel 21 displays a target point and a surrounding region of the target point in a transparent color according to a user's operation, or controls the electrochromic glass plate 24 and the projector 60 such that the projector 60 projects light in a state where the electrochromic glass plate 24 is opaque.

The upper cover 14 has an open upper surface and an open front surface in the form of a quadrangular box to cause the projector 60 to be on an upper space of the housing 11 and serves to block a peripheral view outside the upper space of the housing 11 such that a subject may focus on a target point. The upper cover 14 encloses the housing 11 mounted on an upper plate of the height adjuster 12 and is mounted on the upper plate of the height adjuster 12. That is, the housing 11 is built in a lower portion of an internal space of the upper cover 14. The projector 60 is mounted on a ceiling surface of the internal space of the upper cover 14. It is preferable that the projector 60 be mounted on the ceiling surface of the internal space of the upper cover 14 so as to be perpendicular to the ground.

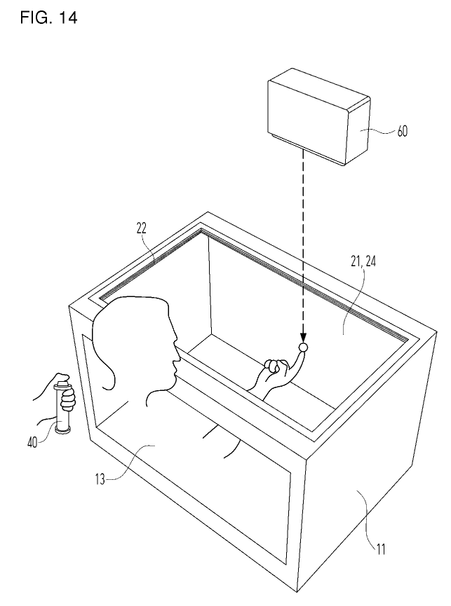

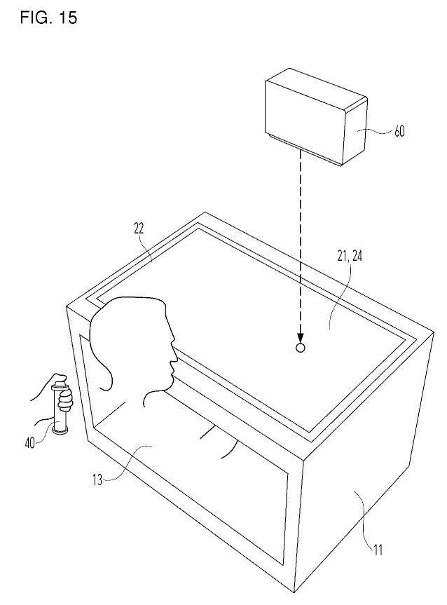

FIGS. 14 and 15 are examples of inputting a reaching point to the proprioception measurement system illustrated in FIGS. 12 and 13. FIG. 14 illustrates a scene in which a reaching point is input to the transparent touch panel 22 by a subject in a state where a surrounding region of a target point displayed on the transparent display panel 21 is transparent and the electrochromic glass plate 24 is transparent. A point touched by a subject is input to the transparent touch panel 22 as a reaching point to reach a target point formed by the light projected from the projector 60 by simultaneously using the subject's visual recognition and proprioception in a state where a surrounding region of the target point is displayed in a transparent color on the transparent display panel 21 and the electrochromic glass plate 24 is in a transparent state such that a movement of the subject's hand located below the transparent touch panel 22 may be visually observed by the subject. In this case, a subject puts his/her hand into the housing 11 and touches the transparent touch panel 22 such that a fingertip reaches a target point while observing a movement of the fingertip utilizing both visual information and proprioception on his/her hand.

FIG. 15 illustrates a scene where a reaching point is input to the transparent touch panel 22 by a subject in a state where a surrounding region of a target point displayed on the transparent display panel 21 is opaque and the electrochromic glass plate 24 is opaque. Because a subject may not observe a finger movement in a state where a surrounding region of a target point is displayed in an opaque color on the transparent display panel 21 and the electrochromic glass plate 24 is opaque such that a movement of the subject's hand located below the transparent touch panel 22 may not be visually observed by the subject, a point touched by the subject is input to the transparent touch panel 22 as a reaching point to reach the target point formed by the light projected from the projector 60 using the subject's proprioception. In this case, the subject puts his/her hand into the housing 11 and touches the transparent touch panel 22 such that a fingertip reaches the target point utilizing only the proprioception of his/her hand while visual information on his/her hand is blocked.

The electrochromic glass plate 24 of the proprioception measurement system illustrated in FIGS. 12 and 13 may be replaced with an opaque film (not illustrated). The opaque film covers an upper surface of the transparent display panel 21 by being displayed on the upper surface of the transparent display panel 21 such that a surrounding region of a target point displayed on the upper surface of the transparent display panel 21 is displayed visually in an opaque state to a subject, or is placed outside a two-dimensional display region of the transparent display panel 21 such that the surrounding region of the target point displayed on the upper surface of the transparent display panel 21 is displayed visually in a transparent state to the subject. In this way, the surrounding region of the target point is displayed in a transparent or opaque state depending on positions of the opaque film.

The projector 60 projects light toward a position, which is preset by a user for an upper surface of an opaque film, under the control of the computer 50 in a state where the opaque film is disposed on an upper surface of the transparent display panel 21, and accordingly, a target point may be formed on the upper surface of the opaque film. A target point is formed on the upper surface of the opaque film by the light projected from the projector 60 in a state where the opaque film is disposed on an upper surface of the transparent display panel 21, and accordingly, the target point that may be visually recognized by a subject may be displayed at a position set by a user. The transparent display panel 21 may or may not display a target point, and the display may also be turned off.

The transparent display panel 21 displays a target point that may be visually recognized by a subject in an opaque color at a position preset by a user on an upper surface under the control of the computer 50 in a state where an opaque film is disposed outside a two-dimensional display region of the transparent display panel 21, and displays a surrounding region of a target point in a transparent color. The computer 50 controls the transparent display panel 21 such that the transparent display panel 21 displays a target point and a surrounding region of the target point in a transparent color according to a user's operation, or causes the projector 60 to project light.

As described above, when opacity of a surrounding region of a target point displayed on the transparent display panel 21 is not sufficient, a subject's visual recognition on the subject's hand movement may be completely blocked by using the electrochromic glass plate 24 or an opaque film. Similarly to the transparent display panel 21 and the transparent touch panel 22, a thickness of the electrochromic glass plate 24 or an opaque film is less, and accordingly, even when an electrochromic glass plate 24 or an opaque film is used, a reaching point is constantly close to a target point. As a result, a subject’s proprioception does not expand, and accordingly, the subject's proprioception may be accurately measured. There are projectors 60 with various resolutions similar to the resolutions of the transparent display panel 21 and the transparent touch panel 22, and accordingly, a subject's proprioception may be easily and precisely improved.

A transparent display panel displays a target point that may be visually recognized by a subject, and a transparent touch panel is disposed under a lower surface of the transparent display panel to detect a reaching point that is a point, which is on the lower surface of the transparent display panel and touched by a subject, to reach a target point displayed on an upper surface of the transparent display panel, and accordingly, a subject’s proprioception may not expand. In this way, because a subject’s proprioception does not expand, the subject's proprioception may be accurately measured. In the conventional proprioception measurement, a distance between a target point and a reaching point causes proprioception corresponding to the distance to expand.

The proprioception may be measured as a subject touches a target point by simultaneously using both visual recognition and proprioception of a subject in a state where a surrounding region of the target point is displayed in a transparent color such that the subject may visually observe a movement of the subject's hand located below a transparent touch panel, and only proprioception may also be measured without visual information as the subject touches the target point using the subject's proprioception in a state where the surrounding region of the target point is displayed in an opaque color such that the movement of the subject's hand located on the lower side of the transparent touch panel may not be visually observed by the subject.

Effects of the disclosure are not limited to the effects described above, and other effects may be derived from the above description.

The disclosure is described with reference to preferred embodiments. Those skilled in the art will appreciate that the disclosure may be implemented in modified forms without departing from the essential characteristics of the disclosure. Therefore, the disclosed embodiments should be considered in an illustrative rather than a restrictive sense. The scope of the disclosure is set forth in the claims, not in the foregoing description, and all differences within the scope equivalent thereto should be construed as being included in the disclosure.

What is claimed is:

1. A proprioception measurement system comprising:
a transparent display panel configured to display a target point, which is visually recognizable by a subject, on an upper surface of the transparent display panel;
a transparent touch panel disposed under a lower surface of the transparent display panel and configured to detect a reaching point, which is touched toward a lower surface of the transparent display panel by the subject to reach a target point displayed on the upper surface of the transparent display panel; and
a computer configured to measure proprioception of the subject based on a display position of the target point displayed on the upper surface of the transparent display panel and a detection position of the reaching point detected by the transparent touch panel.

2. The proprioception measurement system of claim 1, wherein
the transparent touch panel has two surfaces, one surface of which having a touch detection function is disposed facing downward under the lower surface of the transparent display panel, such that a hand of the subject is located in a lower space of the transparent touch panel to input the reaching point to the transparent touch panel in a state where both eyes of the subject look at the upper surface of the transparent display panel.

3. The proprioception measurement system of claim 2, wherein
a point, which is touched by the subject so as to reach the target point simultaneously using both visual recognition and the proprioception of the subject in a state where a surrounding region of the target point is displayed in a transparent color such that a movement of a hand of the subject located below the transparent touch panel is visually observed by the subject, is input to the transparent touch panel as the reaching point.

4. The proprioception measurement system of claim 3, wherein
a point touched by the subject so as to reach the target point using proprioception of the subject in a state where the surrounding region of the target point is displayed in an opaque color such that the movement of the hand of the subject located below the transparent touch panel is not visually observed by the subject, is input to the transparent touch panel as the reaching point.

5. The proprioception measurement system of claim 4, wherein
the transparent display panel has a multilayer structure including a pixel layer composed of multiple pixels, a transparent electrode layer configured to supply power to the multiple pixels, and a transparent substrate layer, and
the surrounding region of the target point is displayed in the transparent color or the opaque color according to color transparency of each of the multiple pixels corresponding to the surrounding region of the target point among the multiple pixels of the pixel layer.

6. The proprioception measurement system of claim 4, further comprising:
an electrochromic glass plate disposed on the upper surface or the lower surface of the transparent display panel and configured to change in transparency according to voltage applied to the electrochromic glass plate,
wherein the surrounding region of the target point is displayed in the transparent color or the opaque color according to the transparency of the electrochromic glass plate.

7. The proprioception measurement system of claim 6, further comprising:
a projector configured to project light toward a position which is preset by a user for an upper surface of the electrochromic glass plate,
wherein the target point is formed and displayed on the upper surface of the electrochromic glass plate by the light projected from the projector in a state where the electrochromic glass plate is opaque.

8. The proprioception measurement system of claim 4, further comprising:
an opaque film disposed on the upper surface of the transparent display panel or disposed outside a two-dimensional display region of the transparent display panel,
wherein the surrounding region of the target point is displayed in the transparent color or the opaque color depending on disposition positions of the opaque film.

9. The proprioception measurement system of claim 8, further comprising:
a projector configured to project light toward a position that is preset by a user for an upper surface of the opaque film,
wherein the target point is formed and displayed on the upper surface of the opaque film by the light projected from the projector in a state where the opaque film is disposed on the upper surface of the transparent display panel.

10. The proprioception measurement system of claim 1, wherein
the computer is further configured to output an error in a detection position of the target point detected by the transparent touch panel relative to a display position of the target point displayed on the upper surface of the transparent display panel as a measurement result of the proprioception of the subject.

11. The proprioception measurement system of claim 1, further comprising:
a housing formed in a shape of a quadrangular box having an open upper surface and an open front surface and configured to provide a space for a lower surface of the transparent touch panel to be touched by a hand of the subject,
wherein the transparent touch panel is mounted on the open upper surface of the housing such that the transparent touch panel is touched by the hand of the subject located in an internal space of the housing through the open front surface of the housing.

12. The proprioception measurement system of claim 11, further comprising:
a height adjustment member installed below a lower surface of the housing to adjust a height of the lower surface of the housing depending on the height of the subject.
